(19) United States
(12) Patent Application Publication
(10) Pub. No.: US 2007/0212358 A1
(43) Pub. Date: Sep. 13, 2007

(54) Compositions and Methods for Effecting Controlled Posterior Vitreous Detachment

(76) Inventor: Stephen P. Bartels, Pittsford, NY (US)

Correspondence Address: Bausch & Lomb Incorporated, One Bausch & Lomb Place, Rochester, NY, 14604-2701, US

(21) Appl. No.: 11/678,222

(22) Filed: Feb. 23, 2007

Related U.S. Application Data
(60) Provisional application No. 60/781,060, filed on Mar. 10, 2006.

(51) Int. Cl. A61K 39/395; A61K 38/54; A61K 38/48; A61K 31/65; A61K 31/19

(52) U.S. Cl. 424/146.1; 424/94.2; 424/94.63; 514/154; 514/575

(57) ABSTRACT

A composition comprises plasmin or an enzymatically equivalent derivative thereof and an inhibitor of at least an enzyme that is activatable, directly or indirectly, by plasmin or one of its enzymatically equivalent derivatives. The composition can be used to effect or induce a controlled posterior vitreous detachment (“PVD”) to prevent, treat, or ameliorate a potential complication of a pathological ocular condition. Such a composition can be administered intravitreally.

### CROSS-REFERENCE

This application claims the benefit of Provisional patent application No. 60/781,060 filed Mar. 10, 2006, which is incorporated by reference herein.

### BACKGROUND OF THE INVENTION

The present invention relates to compositions and methods for effecting controlled posterior vitreous detachment (“PVD”). In particular, the present invention relates to such compositions and methods of treatment that prevent or reduce the potential for their side effects. More particularly, the present invention relates to such compositions comprising plasmin and inhibitors of other proteases and to methods for effecting controlled PVD using such compositions.

Proteases (or proteolytic enzymes or peptidases) are enzymes that catalyze the degradation or breakdown of proteins and, thus, participate in many important physiologic processes. A protease or peptidase can be further classified as an endopeptidase (which cleave peptide bonds within a protein) or exopeptidase (which removes amino acids sequentially from either the N— or the C-terminus of a protein). An endopeptidase is also termed a “proteinase.” Plasmin, a serine proteinase, is the principal fribrinolytic enzyme in mammals, and has the important function of breaking down in-vivo blood clots. It derives from the inactive precursor plasminogen, which circulates in plasma at a concentration of about 1.5 μM. Circulating plasminogen is activated, for example in vivo, by plasminogen activators, such as tissue plasminogen activator (“tPA”) or urokinase, which cleave a single-chain plasminogen molecule at the Arg⁵⁶⁰ -Val⁵⁶¹ peptide bond, producing active plasmin. Plasminogen is also activatable by the bacteria-derived enzyme streptokinase. Thus, thrombolytic drugs, such as those based on tPA, streptokinase, and urokinase, have been developed for administering into patients suffering from various thrombotic disorders, including myocardial infarction, occlusive stroke, deep venous thrombosis, and peripheral arterial disease, to promote the in-vivo production of plasmin in order rapidly to enhance the degradation of blood clots. However, the administered tPA, streptokinase, or urokinase still must encounter the circulating plasminogen in order to generate active plasmin, and the magnitude of the effectiveness of these thrombolytic drugs still depends on the inherent in-vivo level of plasminogen. Therefore, it has been thought that a higher benefit should be obtained if active plasmin is administered instead into these patients.

Plasmin also has been proposed for inducing controlled posterior vitreous detachment (“PVD”) to prevent, stop, or reduce the progression of retinal detachment. U.S. patent application Ser. No. 11/126,625 having the common assignee teaches that creation of a PVD is thought to inhibit the progression of nonproliferative diabetic retinopathy. The references disclosed in that application are incorporated herein by reference.

The vitreous is a clear, proteinaceous mass which fills the posterior cavity of the eye between the lens and the retina. The vitreous is attached at its posterior face to the retina along the structure known as the internal limiting membrane. This site of attachment of the vitreous and the retina is termed the vitreoretinal junction and consists of a layer of basement membrane proximal to the retina and a layer of collagen fibrils proximal to the vitreous.

Degenerative changes in the vitreous are a precursor to PVD. Degeneration of the vitreous is part of the normal aging process, but also may be induced by pathological conditions such as diabetes, Eales' disease and uveitis (see, e.g., “Retinal Detachment” at http://www.emedicine.com/emerg/topic504.html). Because the vitreous is attached to the retina, the receding vitreous can cause a retinal tear, with subsequent detachment of the retina.

Certain pathological conditions of the eye are accompanied by the formation of new (abnormal) membranes in some cases with vessels, i.e. fibrovascular membrane, on the surface of the retina—namely proliferative diseases. With a naturally occurring PVD, traction is placed on these membranes and in those with new vessels there can be rupture and bleeding. Proliferative retinal diseases thus are accompanied by retinal traction and both a high probability of retinal detachment, retinal edema as well as complications from bleeding resulting from the rupture of the newly formed blood vessels. Thus, it is beneficial to induce a controlled PVD before damage to the retina occurs because of uncontrolled detachment. Further, it is thought that attachments between the vitreous and the retina can serve as a scaffold for the abnormal growth of new fibrovascular membranes through the retina and into the vitreous of patients suffering from proliferative back-of-the-eye disorders. Thus, creation of a PVD may avoid or inhibit such growth of fibrovascular membranes into the vitreous.

Verstraeten et al. (Arch. Ophthalmol., Vol. 11, 849-854 (1993)) proposed the use of plasmin to produce a cleavage at the vitreoretinal interface. Plasmin hydrolyzes glycoproteins, including laminin and fibronectin, which are found at the vitreoretinal junction. Plasmin treatment was performed with or without subsequent vitrectomy on rabbit eyes. The authors noted that eyes treated with plasmin showed some areas of PVD, but only after vitrectomy was the vitreous substantially detached. The authors concluded that plasmin treatment may be useful as a biochemical adjunct to mechanical vitrectomy. However, plasmin is a broad-spectrum protease that can participate in other physiologic processes in the eye and thus can potentially produce unintended effects.

Therefore, there is a need to provide compositions comprising plasmin or its enzymatically equivalent derivatives for administration into an eye with at least potentially reduced side effects. In addition, it is also desirable to provide a method for effecting a controlled PVD using such compositions.

### SUMMARY OF THE INVENTION

In general, the present invention provides compositions comprising plasmin or an enzymatically equivalent derivative thereof and one or more inhibitors of other proteases, which compositions can have reduced potential for side effects. The present invention also provides methods for making and using such compositions.

In one aspect, a composition of the present invention comprises plasmin or an enzymatically equivalent derivative thereof and an inhibitor of at least a matrix metalloproteinase (“MMP”), which composition can have reduced potential for side effects when it is administered into an eye.

In another aspect, said at least an MMP is an MMP the pro-enzyme form of which is activatable, directly or indirectly, by plasmin or by said enzymatically equivalent derivative thereof.

In still another aspect, said pro-enzyme form of said MMP is selected from the group consisting of proMMP-1, proMMP-2, proMMP-3, proMMP-7, proMMP-8, proMMP-9, proMMP-10, proMMP-11, proMMP-13, proMMP-14, proMMP-15, combinations thereof, and mixtures thereof.

In yet another aspect, the inhibitor of said at least an MMP is an isolated tissue inhibitor of MMPs (“TIMP”), a synthetic small molecule, a synthetic small molecule capable of binding to the zinc-binding region of said at least an MMP, a chelator of zinc, or an antibody to said at least an MMP.

In still another aspect, the present invention provides a method for effecting a controlled PVD with a reduced potential for side effects. The method comprises administering to or into an eye of a subject in need for said controlled PVD a therapeutically effective amount of a composition that comprises plasmin or one of its enzymatically equivalent derivatives and one or more inhibitors of other proteases, which compositions can have reduced potential for side effects.

In a further aspect, said other proteases comprise MMPs.

In still another aspect, the present invention provides a use of plasmin or one of its enzymatically equivalent derivatives and one or more inhibitors of other proteases for the manufacture of compositions usable for effecting controlled PVD in a subject in need therefor.

Other features and advantages of the present invention will become apparent from the following detailed description and claims and the appended drawings.

### DETAILED DESCRIPTION OF THE INVENTION

In general, the present invention provides compositions comprising plasmin or an enzymatically equivalent derivative thereof and one or more inhibitors of other proteases, which compositions can have reduced potential for side effects.

In one aspect, said other proteases are those that are activatable directly or indirectly by plasmin or one of its enzymatically equivalent derivatives.

In another aspect, said other proteases comprise the family of MMPs.

As used herein, the term “enzymatically equivalent derivative” of plasmin means an enzyme that is derived from plasmin and has a proteolytic function similar to that of plasmin. A derivative of plasmin can be a fragment or a variant of plasmin that has a proteolytic function similar to that of plasmin. A derivative of plasmin can be microplasmin comprising the enzymatic domain of plasmin and a short amino acid sequence (e.g., comprising about 20-40 amino acid residues) at the amino terminus of the enzymatic domain, miniplasmin comprising the enzymatic domain attached to the kringle-5 domain of plasmin, or other truncated forms of plasmin that comprise the enzymatic domain and one or more kringle domains of plasmin having retained lysine-binding property. A variant of plasmin can be generated from a molecule of plasmin by deleting, substituting, or adding one or more amino acid residues. Such substitution can be, for example, a conservative substitution. Enzymatically active microplasmin and miniplasmin are obtained from microplasminogen and miniplasminogen precursors by cleavage of the peptide bond at Arg⁵⁶¹-Val⁵⁶², wherein the amino acid residue numbers correspond to those of human Glu-plasminogen, which has 791 amino acid residues. Microplasmin is disclosed in, for example, U.S. Pat. No. 4,774,087; and miniplasmin is disclosed in, for example, U.S. Patent Application Publications 2005/0118158 and 2005/0124036. The contents of these documents are incorporated herein by reference.

In one aspect, a truncated plasmin comprises the enzymatic domain of plasmin attached at its amino terminus to kringle-1, kringle-2, kringle-3, kringle-4, or kringle-5 domain of plasmin, or combinations thereof. In one embodiment, two or more kringle domains are attached in any order to the amino terminus of the enzymatic domain. A kringle domain of plasmin is characterized by a triple-loop conformation and comprises about 75-85 amino acid residues with three disulfide bridges.

The term “combination” encompasses, but is not limited to, two or more molecules or fragments of molecules attached, attracted, held, or adhered together by bonds (hydrogen bonding, ionic bonding, physical (such as by van der Waals force) or chemical adsorption, covalent bonding, or organometallic interaction), two interpenetrating molecules, or a complex comprising two or more molecules by, e.g., bonding or conformational interaction.

Plasmin is a serine protease that mediates the fribrinolytic process and modulates the extracellular matrix. It hydrolyzes a variety of glycoproteins, including laminin and fibronectin, both of which are present at the vitreoretinal interface and are thought to play a key role in vitreoretinal attachment. Plasmin does not degrade type-IV collagen, a major component of basement membranes and the inner limiting membrane (“ILM”) (see, e.g., A. Gandorfer et al., Investigative Ophthalmology & Visual Science, Vol. 45, No. 2, 641-47 (2004)). Enzymatically equivalent derivatives of plasmin, having the enzymatic domain of plasmin, can thus hydrolyze the same types of polypeptide substrates. Therefore, although the applicants do not wish to be bound by any particular theory, they believe that plasmin and its enzymatically equivalent derivatives hold promise to induce a controlled PVD without damaging the ILM and the retina. Therefore, in one aspect of the present invention, plasmin and/or an enzymatically equivalent derivative thereof can be administered intravitreally to induce a controlled PVD by hydrolyzing selected proteins, including laminin and fibronectin, at the vitreoretinal interface.

However, plasmin is known to activate latent forms of other proteinases, such as MMPs. Active MMPs degrade many extracellular matrix components, including type-IV collagen, entactin, proteoglycans, and glycosaminoglycans (see, e.g.; A. R. Nelson et al., J Clinical Oncology, Vol. 18, No. 5, 1135-49 (2000); M. Mandal et al., Molecular and Cellular Biochemistry, Vol. 252, 303-29 (2003)). These extracellular matrix components are ubiquitously present in many structures of the eye, such as the zonules, the interphoreceptor matrix, and the Bruch's membrane. For example, the ciliary zonules of the eye comprise zonular fibers that comprise parallel bundles of microfibrils, the principal component of which is fibrillin, a glycoprotein. The zonular fibers are surrounded by a coating comprising glycosaminoglycans (such as hyaluronan), proteoglycans (such as chondroitin sulfate and heparin sulfate), and type-IV collagen (L. I. Los et al., J. Histochemistry & Cytochemistry, Vol. 52(6), 789-95 (2004)). Degradation of the proteinaceous coating of the zonular fibers weakens these fibers and can adversely affect the accommodative capability of the lens, and thus the vision. Type-I, -III, -IV, and -V collagen have been identified in the Bruch's membrane (W. S. Karwatowski et al., British J. Ophthalmology, Vol. 79, 944-52 (1995)). Degradation of these types of collagen may promote abnormal subretinal neovascularization, which could lead to vision impairment.

The MMPs comprise a relatively large family of zinc-dependent endopeptidases (or proteinases) and play an important role in tissue remodeling through their ability to degrade the extracellular matrix (“ECM”). Twenty-five MMPs have been identified in various tissues (MMP-1 through MMP-3 and MMP-7 through MMP-28). The proteolytic capacity of MMPs is normally tightly controlled by regulation of the rate of mRNA transcription, by activation of the pro-enzyme, and also by naturally occurring inhibitors such as the tissue inhibitors of MMPs (“TIMPs”) and the plasma protein inhibitor α₂-macroglobulin. MMPs are generally secreted as pro-enzymes that are extracellularly activated by several proteinases. For example, in vitro, plasmin directly activates proMMP-1, proMMP-3, proMMP-7, proMMP-8, proMMP-9, proMMP-10, proMMP-13, and proMMP-14 (see, e.g.; M. Mandal et al., Mol. Cell. Biochem., Vol. 252, 305-29 (2003)). In addition, activation of proMMP-2 involves hydrolysis by MMP-14 (or also known as MT1-MMP), yielding an intermediate that is activated by plasmin. Thus, plasmin most likely plays an important role in the in vivo activation of proMMPs directly or indirectly.

Low levels of several MMPs and their endogenous inhibitors (the tissue inhibitors of MMPs (e.g.; TIMP-1, TIMP-2, and TIMP-3)) are present in the normal vitreous (see, e.g.; J. J. Plantner, Curr. Eye Res., Vol. 17, 132-40 (1998); U.S. Pat. No. 6,787,135), probably serving the function of helping the normal ECM turnover. To date, MMP-1, MMP-2, and MMP-9 have been identified in the vitreous. In view of the capability of MMPs to degrade the ECM, one aspect of the present invention comprises controlling the levels of active MMPs in a method of treatment for inducing a controlled PVD.

Thus, in one aspect, the present invention provides compositions and methods for effecting or inducing a controlled PVD with a reduced potential for side effects, which may be created by, for example, high level of active vitreous MMPs.

In another aspect, the present invention provides compositions for effecting or inducing a controlled PVD, which compositions comprise a therapeutically effective amount of plasmin or an enzymatically equivalent derivative thereof for effecting or inducing a controlled PVD, and an inhibitor of at least an MMP. The amount of the inhibitor of said at least an MMP is enough to inhibit the in-vivo production of said at least an MMP so that its level in the vitreous does not rise substantially above a level of said at least an MMP that is present in a normal vitreous.

In still another aspect, the enzymatically equivalent derivative of plasmin is microplasmin, miniplasmin, or a truncated plasmin. In one embodiment, the truncated plasmin comprises the kringle-1 domain of plasmin attached to the enzymatic domain of plasmin at the amino terminus of the enzymatic domain. In another embodiment, the kringle-1 domain in the truncated plasmin is substituted with the kringle-2, kringle-3, kringle-4, or kringle-5 domain. In still another embodiment, the truncated plasmin comprises two or more, but fewer than five, kringle domains attached in any order to the amino terminus of the enzymatic domain.

Methods for obtaining or producing plasmin and/or its enzymatically equivalent derivative are disclosed below.

In still another aspect, said at least an MMP is an MMP, the pro-enzyme form of which is activatable, directly or indirectly, by plasmin or said one of its enzymatically equivalent derivatives.

In yet another aspect, said pro-enzyme form of said MMP is selected from the group consisting of proMMP-1, proMMP-2, proMMP-3, proMMP-7, proMMP-8, proMMP-9, proMMP-10, proMMP-11, proMMP-13, proMMP-14, proMMP-15, combinations thereof, and mixtures thereof.

In yet another aspect, the inhibitor of said at least an MMP is an isolated tissue inhibitor of MMPs (“TIMP”), a synthetic molecule, a synthetic small molecule capable of binding to the zinc-binding region of said at least an MMP, a chelator of zinc, or an antibody to said at least an MMP.

A tissue inhibitor of MMP can be selected from the group consisting of TIMP-1, TIMP-2, TIMP-3, TIMP-4, and combinations thereof, and mixtures thereof. The TIMPs are available commercially (e.g., from Sigma-Aldrich). They can be isolated from cultures of human fibroblasts using well known methods of cell cultures.

In another aspect, the inhibitor of said at least an MMP is selected from the group consisting of hydroxamate derivatives, carboxylate derivatives, and tetracycline derivatives. Among the peptidic hydroxamate derivatives are Barimastat, Marimastat, and Solimstat developed by British Biotech Pharmaceuticals; the non-peptidic hydroxamate derivatives Prinomastat by Agouron, Rebimastat by Celltech, MMI-270 and CGS27023 by Norvatis, Ro32-3555 by Roche, and RS130,830 by Roche Bioscience; the carboxylic acid-based inhibitors Tanomastat (BAY 12-9566) by Bayer and S-3304 by Shionogi; and the tetracycline derivatives Metastat by CollaGenex and State University of New York, minocycline, and doxycycline.

In yet another aspect, the inhibitor of said at least an MMP is selected from the group consisting of chelators of zinc, such as ethylenediaminetetracetic acid (“EDTA”) or diethylenetriaminepentaacetic acid (“DTPA”).

In a further aspect, the inhibitor of said at least an MMP is an antibody against one of human MMPs. Such an antibody may be raised in rabbits, mice, or sheep, using well-known antibody production methods, and may be further humanized. For example, monoclonal antibodies against human MMP-1, MMP-2, MMP-3, MMP-7, MMP-8, MMP-9, MMP-11, MMP-12, MMP-13, MMP-14, MMP-16, MMP-19, and MMP-20 are commercially available, from, e.g., EMD Biosciences or QED Bioscience Inc.

The concentration of each of plasmin, its enzymatically equivalent derivatives, or one or more of the inhibitors of MMPs in a composition of the present invention can range from about 10₋₄ to about 5, or from about 10⁻³ to about 5, or from about 10⁻² to about 5, or from about 10⁻² to about 2, or from about 10⁻² to about 1 percent by weight.

In one embodiment, a composition of the present invention is in a form of a liquid. In another embodiment, a composition of the present invention is in a form of an aqueous solution. For example, a composition of the present invention can comprise sterile saline solution.

In a further aspect, a composition of the present invention further comprises a compound that has a function of stabilizing plasmin or its enzymatically equivalent derivatives, when present. Such a compound is hereinafter referred to as a “stabilizing agent,” which has a capability of slowing the rate of autodegradation of plasmin or its derivative in a solution; in particular, when the solution has a near neutral pH (e.g., from about 6.5 to about 8.5). The concentration of the stabilizing agent can be in the range from about 0.001 to about 5 weight percent (or alternatively, from about 0.01 to about 4, or from about 0.01 to about 2, or from about 0.01 to about 1 weight percent). The stabilizing agent can be selected from the group consisting of tranexamic acid, ε-aminocaproic acid, L-lysine, analogs of L-lysine, L-arginine, L-ornithine, γ-aminobutyric acid, glycylglycine, gelatin, human serum albumin (“HSA”), glycerin, combinations thereof, and mixtures thereof. Non-limiting examples of analogs of L-lysine include L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

In another aspect, a composition of the present invention can further comprise a non-ionic surfactant, such as polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrji®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., pp 291 and the contents of chapter 22, Lippincott Williams & Wilkins, New York, 2006); the contents of these sections are incorporated herein by reference. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

Plasmin can be produced by activation of plasminogen precursor, which may be obtained from plasma. For example, a method of producing high-purity plasmin is disclosed in U.S. Patent Application Publication 2004/0171103 A1, which is incorporated herein by reference in its entirety. The starting material, plasminogen, can be extracted from Cohn Fraction II+III paste by affinity chromatography on Lys-SEPHAROSE™ as described by D. G. Deutsch and E. T. Mertz, “Plasminogen: purification from human plasma by affinity chromatography,” Science 170(962):1095-6 (1970). (SEPHAROSE™ is a trade name of Pharmacia, Inc., New Jersey.)

Following the extraction of plasminogen from the Cohn Fraction II+III paste, lipid and protein impurities and Transmissible Spongiform Encephalopathies (“TSE”) contaminants are reduced by precipitation with the addition polyethylene glycol (“PEG”), in a range of about 1 to about 10 percent weight/volume or the addition of about 80 to about 120 g/l ammonium sulfate. The PEG or ammonium sulfate precipitate is removed by depth filtration and the resulting solution placed on a lysine affinity resin column. The phrase “lysine affinity resin” is used generally for affinity resins containing lysine or its derivatives or ε-aminocaproic acid as the ligand. The column can be eluted with a solution having a low pH of approximately 1 to 4.

The protein obtained after elution from the affinity column is generally at least 80 percent plasminogen. The purified plasminogen is then stored at low pH in the presence of simple buffers such as glycine and lysine or ω-amino acids.

Plasminogen in solution is then activated to plasmin by the addition of a plasminogen activator, which may be accomplished in a number of ways including but not limited to streptokinase, urokinase, tissue plasminogen activator (“tPA”), or the use of urokinase immobilized on resin and use of streptokinase immobilized on resin. In one embodiment, the plasminogen activator is soluble streptokinase. The addition of stabilizers or excipients such as glycerol, ω-amino acids such as lysine, polylysine, arginine, ε-aminocaproic acid and tranexamic acid, and salt can enhance the yield of plasmin.

Plasmin can be purified from unactivated plasminogen by affinity chromatography on resin with benzamidine as the ligand and eluted preferably with a low pH solution (e.g., pH<4, or alternatively pH between about 2.5 and about 4). This step can remove essentially all degraded plasmin as well as the majority of the streptokinase.

As a polishing step for the removal of remaining streptokinase, hydrophobic interaction chromatography (“HIC”) at low pH is performed (e.g., pH <4). Following the HIC step, plasmin is formulated as a sterile protein solution by ultrafiltration and diafiltration and 0.22-μm filtration.

The eluted plasmin from such polishing step can be buffered with a low pH (e.g., pH<4), low buffering capacity agent. The low pH, low buffering capacity agent typically comprises a buffer of either an amino acid, a derivative of at least one amino acid, an oligopeptide that includes at least one amino acid, or a combination thereof. In addition, the low pH, low buffering capacity agent can comprise a buffer selected from acetic acid, citric acid, hydrochloric acid, carboxylic acid, lactic acid, malic acid, tartaric acid, benzoic acid, serine, threonine, methionine, glutamine, alanine, glycine, isoleucine, valine, alanine, aspartic acid, derivatives, and combinations thereof. The concentration of plasmin in the buffered solution can range from about 0.01 mg/ml to about 50 mg/ml of the total solution. The concentration of the buffer can range from about 1 nM to about 50 mM. Of course, these ranges may be broadened or narrowed depending upon the buffer chosen, or upon the addition of other ingredients such as additives or stabilizing agents. The amount of buffer added is typically that which will give the reversibly inactive acidified plasmin solution at a pH between about 2.5 to about 4, or between about 3 and about 3.5.

It may be advantageous to add a stabilizing or bulking agent to the reversibly inactive acidified plasmin solution obtained as disclosed above. Non-limiting examples of such stabilizing or bulking agents are polyhydric alcohols, pharmaceutically acceptable carbohydrates, salts, glucosamine, thiamine, niacinamide, and combinations thereof. The stabilizing salts can be selected from the group consisting of sodium chloride, potassium chloride, magnesium chloride, calcium chloride, and combinations thereof. Sugars or sugar alcohols may also be added, such as glucose, maltose, mannitol, sorbitol, sucrose, lactose, trehalose, and combinations thereof. Other carbohydrates that may be used are polysaccharides, such as dextrin, dextran, glycogen, starches, carboxymethylcellulose, derivatives thereof, and combinations thereof. Concentrations of a carbohydrate added to add bulk to the reversibly inactive acidified plasmin solution can be in a range from about 0.2 percent weight/volume (“% w/v”) to about 20% w/v. Concentrations for a salt, glucosamine, thiamine, niacinamide, and their combinations can range from about 0.001 M to about 1 M.

Inactive acidified plasmin compositions including a bulking agent, such as a carbohydrate, can be optionally lyophilized at a temperature in a range, for example, from about 0° C. to about −50° C., or preferably from about 0° C. to about −20° C., to produce a powder for long-term storage.

In another aspect, plasmin or variants thereof can be produced by recombinant technology. For example, the production of recombinant microplasminogen (which can be activated to microplasmin by cleavage of the peptide bond at Arg⁵⁶¹-Val⁵⁶² using one of the plasminogen activators disclosed above) in the Pichia pastoris yeast system is disclosed in U.S. Patent Application Publication 2004/0071676 A1, which is incorporated herein by reference. Plasminogen and miniplasminogen (which also can be activated to miniplasmin by cleavage of the peptide bond at Arg⁵⁶¹-Val⁵⁶² using one of the plasminogen activators disclosed above) in the Pichia pastoris yeast system are disclosed in U.S. Patent Application Publication 2005/0124036 A1, which is incorporated herein by reference.

Recombinant plasmin or variants thereof are acidified and stored at pH less than about 5 (or alternatively less than about 4, or between about 2.5 and about 3.5). The acidified plasmin or variants thereof thus produced can further be lyophilized for long-term storage.

In one aspect, the acidified plasmin or variants thereof, produced from plasma or by recombinant technology, can be reconstituted by adding the enzyme to a formulation having a near neutral pH, to produce a formulated enzyme substantially immediately before using the enzyme.

In another aspect, the formulation has a pH of about 7. Alternatively, the formulation has a pH in a range from about 7 to about 7.5.

In still another aspect, the formulation has a pH of about 7.4.

In yet another aspect, the formulation comprises a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5× PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl)-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pkₐ of 7.52 at 25° C. and pH in the range of about 7-8.2; TAPSO (2-hydroxy-3{tris(hydroxymethyl)methylamino}-1propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethansulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In one aspect, the pH of the formulation is in the range from about 6.5 to about 11. Alternatively, the pH of the formulation is in the range from about 6.5 to about 9, or from about 6.5 to about 8. In another aspect, the formulation comprises a buffer having a pH in one of said pH ranges.

In another aspect, the present invention provides a method for producing a composition for use in inducing a controlled PVD, the method comprising adding plasmin or an enzymatically equivalent thereof with an inhibitor of at least another enzyme, the latent form of which is activatable by plasmin or by said enzymatically equivalent thereof. In one embodiment, said at least another enzyme is an MMP. In one embodiment, the mixing is carried out in a medium comprising a buffer having a pH in the range from about 6.5 to about 8.5.

In another aspect, the present invention provides a method for producing a composition for use in inducing a controlled PVD, the method comprising: (a) storing plasmin or an enzymatically equivalent derivative thereof at a pH less than about 5; and (b) adding said stored plasmin or enzymatically equivalent derivative thereof to a formulation that comprises an inhibitor of at least another enzyme, the latent form of which is activatable, directly or indirectly, by plasmin or by said enzymatically equivalent derivative thereof. In one embodiment, said at least another enzyme is an MMP.

In a further aspect, an inhibitor of at least an MMP is selected from the group of inhibitors of MMPs disclosed above.

In one embodiment, the formulation further comprises a buffer having a pH in the range from about 6.5 to about 11 (or alternatively, from about 6.5 to about 9, or from about 6.5 to about 8).

In another aspect, the formulation further comprises a compound selected from the group consisting of tranexamic acid, ε-aminocaproic acid, L-lysine, analogs of L-lysine, L-arginine, L-ornithine, γ-aminobutyric acid, glycylglycine, gelatin, HSA, glycerin, combinations thereof, and mixtures thereof. Non-limiting examples of analogs of L-lysine include L-2-amino-3-guanidinopropionic acid, L-citruline, D-citruline, 2,6-diaminoheptanoic acid, ε,ε-dimetyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl-D-ornithine, p-aminomethylbenzoic acid, and 2-aminoethylcysteine.

In another embodiment, the formulation further comprises a non-ionic surfactant. Non-limiting examples of suitable non-ionic surfactants are disclosed above.

In another aspect, the step of storing of said enzyme is effected at a pH less than about 4.5. Alternatively, said pH is less than 4 or in the range from about 2.5 to about 4.5, or from about 2.5 to about 4, or from about 3 to about 4.

In still another aspect, the present invention is useful in producing a composition comprising active plasmin or an enzymatically equivalent derivative thereof after prolonged storage after its manufacture for use in inducing a controlled PVD in a patient with at least a reduced potential for side effects.

In still another aspect, the present invention provides a kit for making a composition for use in inducing a controlled PVD. The composition comprises plasmin or an enzymatically equivalent derivative thereof and an inhibitor of at least another enzyme, the latent form of which is activatable, directly or indirectly, by plasmin or by said enzymatically equivalent derivative thereof. The kit comprises: (a) plasmin or said enzymatically equivalent derivative thereof that has been preserved at a pH less than about 5; and (b) a formulation that comprises said inhibitor of said at least another enzyme, said formulation being provided in a separate container or package. In one embodiment, said another enzyme is an MMP. In another embodiment, said inhibitor is selected from the groups of MMP inhibitors disclosed above.

In still another embodiment, the formulation further comprises a compound selected from the group consisting of tranexamic acid, ε-aminocaproic acid, L-lysine, analogs of L-lysine, L-arginine, L-ornithine, γ-aminobutric acid, glycylglycine, gelatin, HSA, glycerin, combinations thereof, and mixtures thereof. Non-limiting examples of analogs of L-lysine include L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

In still another aspect, the present invention provides a method for inducing a controlled PVD in an eye of a patient, the method comprising: (a) providing a composition that comprises plasmin or an enzymatically equivalent derivative thereof and an inhibitor of at least another enzyme, a latent form of which is activatable, directly or indirectly, by plasmin or by said enzymatically equivalent derivative thereof, said at least another enzyme being present in a normal vitreous; and (b) administering said composition into the posterior chamber of the eye, thereby inducing said controlled PVD in said eye. In one embodiment, said another enzyme is an MMP. In another embodiment, said another enzyme is MMP-1, MMP-2, MMP-3, MMP-7, MMP-8, MMP-9, MMP-10, MMP-11, MMP-13, MMP-14, MMP-15, combinations thereof, or mixtures thereof.

In one embodiment, the composition further comprises a buffer having a pH in the range from about 6.5 to about 11 (or alternatively, from about 6.5 to about 9, or from about 6.5 to about 8).

In another embodiment, the composition further comprises a compound selected from the group consisting of tranexamic acid, ε-aminocaproic acid, L-lysine, analogs of L-lysine, L-arginine, L-ornithine, γ-aminobutyric acid, glycylglycine, gelatin, HSA, glycerin, combinations thereof, and mixtures thereof. Non-limiting examples of analogs of L-lysine include L-2-amino-3-guanidinopropionic acid, L-citruline, D-citruline, 2,6-diaminoheptanoic acid, ε,ε-dimethyl-L-lysine, α-methyl-DL-ornithine, 5-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

Non-limiting amounts or concentrations of the various materials or compounds disclosed above are also applicable to the various methods of the present invention disclosed herein.

In a further aspect, the step of providing said composition comprises: (i) providing said plasmin or said enzymatically equivalent derivative thereof that has been preserved at a pH less than about 5; (ii) providing said inhibitor of said at least another enzyme; and (iii) producing said composition from said plasmin or said enzymatically equivalent derivative thereof and said inhibitor of said at least another enzyme.

In still another aspect, the patient may be one who has one or more symptoms of the beginning of a pathological PVD and the method induces a controlled PVD. Such a controlled PVD can arrest or prevent damage to the retina, which would occur if the pathological uncontrolled PVD is allowed to continue.

In another embodiment, said composition is administered in an amount containing a therapeutically effective amount of plasmin or an enzymatically equivalent derivative thereof to induce said controlled PVD.

Method of injecting plasmin or derivatives thereof into eye for controlled PVD is now described.

A composition comprising plasmin or an enzymatically equivalent derivative thereof and an inhibitor of at least another enzyme that is activatable, directly or indirectly, by plasmin or said enzymatically equivalent derivative thereof can be injected intravitreally, for example through the pars plana of the ciliary body, to induce controlled PVD using a fine-gauge needle, such as 25-30 gauge. Administration of such a composition can be used to prevent, treat, or ameliorate the potentially blinding complications of an ocular condition, such as diabetic retinopathy, retinal detachment, macular edema, macular hole, and retinal tears. Typically, an amount from about 25 μl to about 200 μl of a composition comprising about 1-5 IU of plasmin or derivatives thereof per 50 μl of formulation is administered into the vitreous. Alternatively, a composition can comprise about 0.001-50 mg/ml (or about 0.2-20 mg/ml, or about 0.2-10 mg/ml, or about 0.5-8 mg/ml) of plasmin or derivatives thereof. Such administration of plasmin or derivatives thereof may repeated to achieve a substantially full effect upon assessment of the treatment results and recommendation by a skilled medical practitioner.

Tables 1-16 show non-limiting examples of compositions of the present invention, which can be used in the practice of the methods of the present invention disclosed above.

TABLE 1

| Ingredient | Amount per ml | | % composition |
|---|---|---|---|
| plasmin | 2 | mg | 0.2 |
| trehalose | 20 | mg | 2 |
| sodium acetate | 2.4 | mg | 0.24 |
| ε-amino caproic acid | 3 | mg | 0.3 |
| TIMP-1 | 2 | mg | 0.2 |
| normal saline | QS to 1 | ml | 97.06 |

TABLE 2

| Ingredient | Amount per ml | | % composition |
|---|---|---|---|
| plasmin | 2 | mg | 0.2 |
| trehalose | 20 | mg | 2 |
| sodium acetate | 2.4 | mg | 0.24 |
| ε-amino caproic acid | 3 | mg | 0.3 |
| TIMP-2 | 2 | mg | 0.2 |
| normal saline | QS to 1 | ml | 97.06 |

TABLE 3

| Ingredient | Amount per ml | | % composition |
|---|---|---|---|
| plasmin | 4 | mg | 0.4 |
| trehalose | 20 | mg | 2 |
| sodium acetate | 2.4 | mg | 0.24 |
| ε-amino caproic acid | 3 | mg | 0.3 |
| TIMP-3 | 2 | mg | 0.2 |
| normal saline | QS to 1 | ml | 96.86 |

TABLE 4

| Ingredient | Amount per ml | | % composition |
|---|---|---|---|
| plasmin | 2 | mg | 0.2 |
| trehalose | 20 | mg | 2 |
| sodium acetate | 2.4 | mg | 0.24 |
| tranexamic acid | 3 | mg | 0.3 |
| TIMP-1 | 2 | mg | 0.2 |
| TIMP-2 | 2 | mg | 0.2 |
| TIMP-3 | 2 | mg | 0.2 |
| normal saline | QS to 1 | ml | 96.66 |

TABLE 5

| Ingredient | Amount per ml | | % composition |
|---|---|---|---|
| plasmin | 10 | mg | 1 |
| trehalose | 20 | mg | 2 |
| sodium acetate | 2.4 | mg | 0.24 |
| ε-amino caproic acid | 3 | mg | 0.3 |
| Barimastat | 5 | mg | 0.5 |
| normal saline | QS to 1 | ml | 95.96 |

TABLE 6

| Ingredient | Amount per ml | | % composition |
|---|---|---|---|
| microplasmin | 2 | mg | 0.2 |
| trehalose | 20 | mg | 2 |
| sodium acetate | 2.4 | mg | 0.24 |
| ε-amino caproic acid | 3 | mg | 0.3 |
| Marimastat | 3 | mg | 0.3 |
| normal saline | QS to 1 | ml | 96.96 |

TABLE 7

| Ingredient | Amount per ml | | % composition |
|---|---|---|---|
| miniplasmin | 2 | mg | 0.2 |
| trehalose | 20 | mg | 2 |
| sodium citrate | 2.4 | mg | 0.24 |
| tranexamic acid | 3 | mg | 0.3 |
| Solimstat | 5 | mg | 0.5 |
| normal saline | QS to 1 | ml | 96.76 |

TABLE 8

| Ingredient | Amount per ml | | % composition |
|---|---|---|---|
| a truncated plasmin | 2 | mg | 0.2 |
| consisting essentially | | | |
| of kringle-1 domain | | | |
| and enzymatic | | | |
| domain of plasmin | | | |
| mannitol | 20 | mg | 2 |
| sodium acetate | 2.4 | mg | 0.24 |
| ε-amino caproic acid | 3 | mg | 0.3 |
| Rebimastat | 5 | mg | 0.5 |
| normal saline | QS to 1 | ml | 96.76 |

TABLE 9

| Ingredient | Amount per ml | | % composition |
|---|---|---|---|
| plasmin | 2 | mg | 0.2 |
| trehalose | 20 | mg | 2 |
| sodium acetate | 2.4 | mg | 0.24 |
| ε-amino caproic acid | 3 | mg | 0.3 |
| MMI-270 | 2 | mg | 0.2 |
| normal saline | QS to 1 | ml | 97.06 |

TABLE 10

| Ingredient | Amount per ml | | % composition |
|---|---|---|---|
| plasmin | 2 | mg | 0.2 |
| trehalose | 20 | mg | 2 |
| sodium acetate | 2.4 | mg | 0.24 |
| ε-amino caproic acid | 3.0 | mg | 0.3 |
| CGS27023 | 3 | mg | 0.3 |
| normal saline | QS to 1 | ml | 96.96 |

TABLE 11

| Ingredient | Amount per ml | | % composition |
|---|---|---|---|
| plasmin | 2 | mg | 0.2 |
| mannitol | 20 | mg | 2 |
| sodium acetate | 2.4 | mg | 0.24 |
| ε-amino caproic acid | 3.0 | mg | 0.3 |
| BAY 12-9566 | 6 | mg | 0.6 |
| normal saline | QS to 1 | ml | 96.66 |

TABLE 12

| Ingredient | Amount per ml | | % composition |
|---|---|---|---|
| plasmin | 2 | mg | 0.2 |
| trehalose | 20 | mg | 2 |
| sodium acetate | 2.4 | mg | 0.24 |
| ε-amino caproic acid | 3 | mg | 0.3 |
| rosiglitazone | 2 | mg | 0.2 |
| Ro32-3555 | 5 | mg | 0.5 |
| normal saline | QS to 1 | ml | 96.56 |

TABLE 13

| Ingredient | Amount per ml | | % composition |
|---|---|---|---|
| miniplasmin | 2 | mg | 0.2 |
| trehalose | 20 | mg | 2 |
| sodium acetate | 2.4 | mg | 0.24 |
| ε-amino caproic acid | 3 | mg | 0.3 |
| RS130,830 | 5 | mg | 0.5 |
| Ro32-3555 | 3 | mg | 0.3 |
| normal saline | QS to 1 | ml | 96.46 |

TABLE 14

| Ingredient | Amount per ml | | % composition |
|---|---|---|---|
| microplasmin | 5 | mg | 0.5 |
| trehalose | 20 | mg | 2 |
| sodium citrate | 2.4 | mg | 0.24 |
| tranexamic acid | 3 | mg | 0.3 |
| RS130,830 | 5 | mg | 0.5 |
| BAY 12-9566 | 3 | mg | 0.3 |
| phosphate buffer (pH | QS to 1 | ml | ~96.1 |
| 7.4) | | | |

TABLE 15

| Ingredient | Amount per ml | | % composition |
|---|---|---|---|
| miniplasmin | 2 | mg | 0.2 |
| mannitol | 20 | mg | 2 |
| sodium acetate | 2.4 | mg | 0.24 |
| ε-amino caproic acid | 3 | mg | 0.3 |
| BAY 12-9566 | 5 | mg | 0.5 |
| S-3304 | 3 | mg | 0.3 |
| normal saline | QS to 1 | ml | 96.46 |

TABLE 16

| Ingredient | Amount per ml | | % composition |
|---|---|---|---|
| plasmin | 2 | mg | 0.2 |
| mannitol | 20 | mg | 2 |
| sodium acetate | 2.4 | mg | 0.24 |
| L-lysine | 2 | mg | 0.2 |
| Metastat | 5 | mg | 0.5 |
| minocycline | 3 | mg | 0.3 |
| doxycycline | 1 | mg | 0.1 |
| normal saline | QS to 1 | ml | 96.46 |

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising: (a) plasmin or an enzymatically equivalent derivative thereof; and (b) at least an inhibitor of another enzyme, a pro-enzyme form of which is activatable, directly or indirectly, by said plasmin or said enzymatically equivalent derivative thereof.

2. The composition of claim 1, wherein said another enzyme comprises a matrix metalloproteinase (“MMP”).

3. The composition of claim 2, wherein said another enzyme is selected from the group consisting of MMP-1, MMP-2, MMP-3, MMP-7, MMP-8, MMP-9, MMP-10, MMP-11, MMP-13, MMP-14, MMP-15, combinations thereof, and mixtures thereof.

4. The composition of claim 2, wherein said inhibitor of said another enzyme is selected from the group consisting of tissue inhibitors of MMPs (“TIMPs”), synthetic molecules capable of binding to the zinc-binding domain of an MMP, hydroxamate derivatives, carboxylate derivatives, tetracycline derivatives, chelators of zinc, antibodies against an MMP, combinations thereof, and mixtures thereof.

5. The composition of claim 2, wherein the enzymatically equivalent derivative of plasmin is selected from the group consisting of microplasmin, miniplasmin, truncated forms of plasmin, variants of plasmin, combinations thereof, and mixtures thereof.

6. The composition of claim 2, wherein the composition further comprises a stabilizing agent for said plasmin or said enzymatically equivalent derivative thereof.

7. The composition of claim 6, wherein the stabilizing agent is selected from the group consisting of tranexamic acid, ε-aminocaproic acid, L-lysine, analogs of L-lysine, L-arginine, L-ornithine, γ-aminobutyric acid, glycylglycine, gelatin, human serum albumin (“HSA”), glycerin, combinations thereof, and mixtures thereof.

8. The composition of claim 7, wherein the L-lysine analogs are selected from the group consisting of L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

9. The composition of claim 2, wherein said inhibitor of said another enzyme is selected from the group consisting of hydroxamate derivatives, carboxylate derivatives, combinations thereof, and mixtures thereof.

10. The composition of claim 2, wherein said inhibitor of said another enzyme is selected from the group consisting of Barimastat, Marimastat, Solimstat, Prinomastat, Rebimastat, MMI-270, CGS27023, Ro32-3555, RS130,830, Tanomastat (BAY 12-9566), S-3304, Metastat, tetracycline derivatives, minocycline, doxycycline, combinations thereof, and mixtures thereof.

11. The composition of claim 2, wherein said inhibitor of said another enzyme is selected from the group consisting of ethylenediaminetetraacetic acid (“EDTA”), ditehylenetriaminepentaacetic acid (“DTPA”), combinations thereof, and mixtures thereof.

12. A composition comprising: (a) plasmin or an enzymatically equivalent derivative thereof; and (b) at least an inhibitor of an MMP, a pro-enzyme form of said MMP being activatable, directly or indirectly, by said plasmin or said enzymatically equivalent derivative thereof, wherein said at least an inhibitor of an MMP is selected from the group consisting of TIMPs, synthetic molecules capable of binding to the zinc-binding domain of an MMP, chelators of zinc, antibodies against an MMP, hydroxamate derivatives, carboxylate derivatives, tetracycline derivatives, combinations thereof, and mixtures thereof.

13. The composition of claim 12, wherein a concentration of each of said plasmin, said enzymatically equivalent derivative of plasmin, and said inhibitor is in a range from about 10⁻⁴ to about 5 weight percent.

14. The composition of claim 13, further comprising a stabilizing agent for plasmin or an enzymatically equivalent derivative thereof.

15. A method for producing a composition for use in inducing a controlled posterior vitreous detachment (“PVD”), the method comprising:
(a) providing plasmin or an enzymatically equivalent derivative thereof; and
(b) adding said plasmin or enzymatically equivalent derivative thereof to an inhibitor of at least another enzyme, a latent form of which is activatable, directly or indirectly, by said plasmin or enzymatically equivalent derivative thereof.

16. The method of claim 15, wherein said plasmin or an enzymatically equivalent derivative thereof has been preserved at a pH less than about 5.

17. The method of claim 15, further comprising adding a stabilizing agent selected from the group consisting of tranexamic acid, ε-aminocaproic acid, L-lysine, analogs of L-lysine, L-arginine, L-ornithine, γ-aminobutyric acid, glycylglycine, gelatin, HSA, glycerin, combinations thereof, and mixtures thereof.

18. The method of claim 15, wherein said at least another enzyme is selected from the group of MMPs.

19. The method of claim 15, wherein said inhibitor of at least another enzyme is selected from the group consisting of TIMPs, synthetic molecules capable of binding to the zinc-binding domain of an MMP, hydroxamate derivatives, carboxylate derivatives, tetracycline derivatives, chelators of zinc, antibodies against an MMP, combinations thereof, and mixtures thereof.

20. Use of plasmin or an enzymatically equivalent derivative thereof and an inhibitor of at least another enzyme, a latent form of which is activatable, directly or indirectly, by said plasmin or enzymatically equivalent derivative thereof, to produce a composition for inducing a controlled PVD in a subject in need therefor.

21. The use of claim 20, wherein said inhibitor of at least another enzyme is selected from the group consisting of TIMPs, synthetic molecules capable of binding to the zinc-binding domain of an MMP, hydroxamate derivatives, carboxylate derivatives, tetracycline derivatives, chelators of zinc, antibodies against an MMP, combinations thereof, and mixtures thereof.

22. A method for inducing a controlled PVD in an eye of a patient, the method comprising:
(a) providing a composition that comprises plasmin or an enzymatically equivalent derivative thereof and an inhibitor of at least another enzyme, a latent form of which is activatable, directly or indirectly, by plasmin or by said enzymatically equivalent derivative thereof, said at least another enzyme being present in a normal vitreous; and
(b) administering said composition into the vitreous humor of the eye, thereby inducing said controlled PVD in said eye.

23. The method of claim 22, wherein said another enzyme comprises an MMP.

24. The method of claim 22, wherein said another enzyme is selected from the group consisting of MMP-1, MMP-2, MMP-3, MMP-7, MMP-8, MMP-9, MMP-10, MMP-11, MMP-13, MMP-14, MMP-15, combinations thereof, and mixtures thereof.

25. The method of claim 22, wherein said inhibitor of said another enzyme is selected from the group consisting of TIMPs, synthetic molecules capable of binding to the zinc-binding domain of an MMP, hydroxamate derivatives, carboxylate derivatives, tetracycline derivatives, chelators of zinc, antibodies against an MMP, combinations thereof, and mixtures thereof.

26. The method of claim 22, wherein said inhibitor of said another enzyme is selected from the group consisting of TIMPs, hydroxamate derivatives, carboxylate derivatives, tetracycline derivatives, antibodies against an MMP, combinations thereof, and mixtures thereof.

27. The method of claim 22, wherein said enzymatically equivalent derivative of plasmin is selected from the group consisting of microplasmin, miniplasmin, truncated forms of plasmin, combinations thereof, and mixtures thereof.

28. The method of claim 22, wherein the composition further comprises a stabilizing agent for said plasmin or said enzymatically equivalent derivative thereof.

29. The method of claim 22, wherein said plasmin or enzymatically equivalent derivative thereof has been preserved at a pH less than about 5.

30. The method of claim 22, wherein said controlled PVD is induced to prevent, treat, or ameliorate at least a potential complication of an ocular condition selected from the group consisting of vitreoretinal traction, diabetic retinopathy, retinal detachment, macular edema, macular hole, epiretinal membrane, macular pucker and retinal tears.

31. The method of claim 22, wherein said composition is administered in an amount sufficient to induce said controlled PVD.

32. A kit for producing a composition useful for inducing a controlled PVD, the kit comprising:
(a) plasmin or an enzymatically equivalent derivative thereof disposed in a first container; and
(b) an inhibitor of at least another enzyme that is activatable, directly or indirectly, by plasmin or an enzymatically equivalent derivative thereof, disposed in a second container, wherein contents of said first and second containers are combined to produce said composition.

33. The kit of claim 32, wherein said another enzyme comprises a matrix metalloproteinase (“MMP”).

34. The kit of claim 33, wherein said another enzyme is selected from the group consisting of MMP-1, MMP-2, MMP-3, MMP-7, MMP-8, MMP-9, MMP-10, MMP-11, MMP-13, MMP-14, MMP-15, combinations thereof, and mixtures thereof.

35. The kit of claim 33, wherein said inhibitor of said another enzyme is selected from the group consisting of TIMPs, synthetic molecules capable of binding to the zinc-binding domain of an MMP, hydroxamate derivatives, carboxylate derivatives, tetracycline derivatives, chelators of zinc, antibodies against an MMP, combinations thereof, and mixtures thereof.

36. The kit of claim 33, wherein the enzymatically equivalent derivative of plasmin is selected from the group consisting of microplasmin, miniplasmin, truncated forms of plasmin, variants of plasmin, combinations thereof, and mixtures thereof.

37. The kit of claim 33, wherein said second container further contains a stabilizing agent for said plasmin or said enzymatically equivalent derivative thereof.
